(19) United States
(12) Plant Patent Application Publication
(10) Pub. No.: US 2014/0223627 P1
(43) Pub. Date: Aug. 7, 2014

(54) Dendrobium orchid plant 'SPCDW1004'

(50) ;

(71) Applicant: Thumrong Suphachadiwong, Chonburi (TH)

(72) Inventor: Thumrong Suphachadiwong, Chonburi (TH)

(73) Assignee: Suphachatwong Innovation Co, Chonburi (TH)

(21) Appl. No.: 13/999,299

(22) Filed: Feb. 6, 2014

(30) Foreign Application Priority Data
Feb. 7, 2013 (QZ) A201300293

(51) Int. Cl. A01H 5/00

(52) U.S. Cl. PLT/311

(57) ABSTRACT

A new and distinct Dendrobium cultivar named ‘SPCDW1004’ is disclosed, characterized by its large pure white flower. The new variety is a Dendrobium, typically produced as an indoor ornamental plant.

### LATIN NAME OF THE GENUS AND SPECIES

Dendrobium hybrid.

### VARIETY DENOMINATION

‘SPCDW1004’

### BACKGROUND OF THE INVENTION

This application relates to a new cultivar of Dendrobium hybrid. The new variety is the product of a planned breeding program. The new variety originated as a seedling from the crossing of the unpatented, proprietary seed parent Dendrobium ‘SK151’ with the unpatented, proprietary pollen parent referred to as Dendrobium ‘PTW’. The crossing was made by the inventor in his research greenhouse in Chonburi, Thailand in October of 2000.

The new variety was first selected by the inventor, Thumrong Suphachadiwong, a citizen of Thailand, in March of 2006 in a research greenhouse belonging to the inventor in Chonburi, Thailand. After identifying the new variety as a potentially interesting selection, the inventor continued confidential testing and propagation of ‘SPCDW1004’, assessing stability of the unique characteristics of this variety.

Asexual reproduction of the new cultivar ‘SPCDW1004’ was first performed at a commercial laboratory belonging to the inventor in Chonburi, Thailand by tissue culture in March of 2006. Access to all plants was restricted, as plants were kept in a greenhouse not open to the public, and tissue culture plantlets were in a laboratory belonging to the inventor, and not accessible to the public. Through subsequent propagation, multiple generations have been reproduced, which have shown that the unique features of this cultivar are stable and reproduced true to type.

### SUMMARY OF THE INVENTION

The cultivar ‘SPCDW1004’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘SPCDW1004.’ These characteristics in combination distinguish ‘SPCDW1004’ as a new and distinct Dendrobium cultivar:
1. Pure white flowers.
2. Large flower size.

### Parent Comparison

Plants of the new cultivar ‘SPCDW1004’ are similar to the seed parent, Dendrobium ‘SK151’ in most horticultural characteristics. However the new variety, ‘SPCDW1004’ differs from ‘SK151’ in the following characteristics:
1. Parent has light pink flowers ‘SPCDW1004’ has pure white flower.

Plants of the new cultivar ‘SPCDW1004’ are similar to the pollen parent, Dendrobium ‘PTW’ in most horticultural characteristics. However the new variety, ‘SPCDW1004’ differs from ‘PTW’ in the following characteristics:
1. Spikes produce more flowers and grow more upright.
2. More compact plant.
3. Smaller flower size.

### Commercial Comparison

‘SPCDW1004’ is similar in most horticultural characteristics to the unpatented commercial variety Dendrobium ‘White Surprise’ in most horticultural characteristics. However the new variety, ‘SPCDW1004’ differs from ‘White Surprise’ in the following characteristics:
1. Smaller flower size.
b 2. Shorter plant height.
3. Narrower sepals.
4. Different column color.

Plants of the new cultivar ‘SPCDW1004’ are also comparable to the unpatented commercial variety Dendrobium ‘Snow Jade’. The two Dendrobium varieties are similar in most horticultural characteristics; however, the new variety ‘SPCDW1004’ differs in the following ways:
1. Different lip shape.
2. Narrower dorsal and lateral sepal shape.
3. Pure white flower. The comparator flowers are greenish white.

### BRIEF DESCRIPTION OF THE PHOTOGRAPH

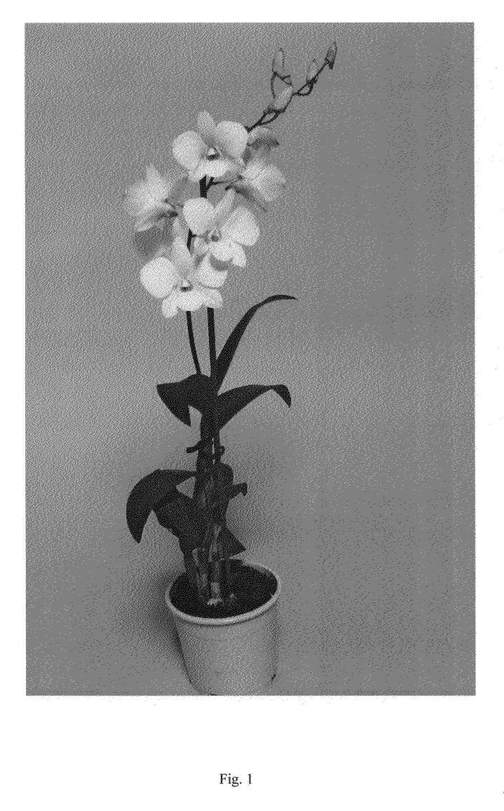

The accompanying photograph in FIG. 1 illustrates in full color a typical plant of ‘SPCDW1004’ grown in a commercial greenhouse in Honselersdijk, the Netherlands. The plant is 1 years old from a rooted cutting, in a 10.5 cm pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007, except where general terms of ordinary dictionary significance are used. The following observations and measurements describe ‘SPCDW1004’ plants grown in a commercial greenhouse in Honselersdijk, the Netherlands. The plant is 1 year old from a rooted cutting, in a 10.5 cm pot. Temperatures ranged between 26° C. and 30° C. day and night. No photoperiodic or chemical treatments were given to the plants. The light sum was set at 5 moles per day and the plant was given shading and artificial light in order to come to the light sum of 5 moles per day. Both shading and artificial lightning were frequently applied in order to achieve the ideal light sum of 5 moles per day. Measurements and numerical values represent averages of typical plant types.
Botanical classification: Dendrobium hybrid ‘SPCDW1004’
Propagation:
Type of propagation typically used: Tissue culture.
Time to initiate roots: Tissue culture takes 18 months. After deflasking it takes another 9 to 12 months to get a well-rooted plant under average temperatures ranging between 26 en 30 degrees Celsius. After planting in the end pot, the young plant already has roots and those roots continue to grow in the end pot almost right away and certainly within 8 days.
Time to produce a rooted cutting/liner: Tissue culture takes 18 months. After deflasking it takes another 9 to 12 months to get a well-rooted plant under average temperatures ranging between 26 en 30 degrees Celsius. After planting in the end pot, the young plant already has roots and those roots continue to grow in the end pot almost right away and certainly within 8 days.
Root description:
   Root color.—Yellow-white near RHS 158D.
   Average diameter.—Average: 0.2 cm.
   Average length.—Lateral roots average: 4.8 cm.
Plant:
Growth habit: Sympodial, upright
Height from top of soil line to top of foliage: 31.0 cm
Height from top of soil line to top of flowers: 48.5 cm
Plant spread: 21.5 cm
Normal pot size: 10.5 cm circular pot
Growth rate: Moderate
Number of leaves per plant: Average: 12
Plant stem:
   Length.—Approximately 13.2 cm.
   Diameter.—Approximately 1.7 cm.
   Aspect.—Approximately 75° angle to soil surface (=0°).
   Strength.—Strong.
   Texture.—Glabrous, very slightly glossy, axially ribbed.
   Color.—Yellow-green, in between near RHS N144A and 152C.
Foliage:
Leaf:
   Arrangement.—Distichous.
   Average length.—Approximately 12.5 cm.
   Average width.—Approximately 4.3 cm.
   Shape of blade.—Narrow Ovate to elliptic.
   Aspect.—Leaves in an average angle of 70° to main stem (=0°).
   Apex.—Acute.
   Base.—Sheathing Sheath length: Average 3.0 cm Sheath width: Average 1.6 cm. Sheath color: Yellow-green, near RHS 147D, slightly striped axially, yellow-green; near RHS 147B, stripes coloured purple near the base; near RHS N79A.
   Margin.—Entire.
   Texture of top surface.—Glabrous, moderately glossy.
   Texture of bottom surface.—Glabrous, matte.
   Durability to physical stress.—High.
   Color.—Young foliage upper side: Green; in between near RHS 137A and 143A Young foliage under side: Yellow-green; near RHS 147B Mature foliage upper side: Green; in between near RHS N137A and 143A but darker Mature foliage under side: Yellow-green; near RHS 146A to 146B.
   Venation.—Type: Parallel Venation coloration: Upper Surface: Green; in between near RHS N137A and 143A but darker Lower Surface: Yellow-green; near RHS 146B.
   Petiole.—No petioles present.
Flowering characteristics:
Flower bud:
   Rate of opening.—Approximately 30 days from first sight.
   Length.—Approximately 2.4 cm.
   Diameter.—Approximately 1.1 cm.
   Shape.—Ovate, curved.
   Color.—Yellow-green; near 145B, tip of spur purple; near RHS N77B.
Peduncle:
   Length.—Approximately 26.0 cm.
   Diameter.—Approximately 0.3 cm.
   Aspect.—Approximately 0° angle to stem.
   Strength.—Strong.
   Texture.—Glabrous, slightly glossy.
   Color.—Yellow-green; near RHS 144A, sunny side tinged darker; near RHS 146A.
Inflorescence:
   Flowers per stem.—Average: 10.
   Inflorescence arrangement.—Simple raceme.
   Inflorescence height.—Approximately 17.5 cm (from lowest pedicel to top of inflorescence).
   Inflorescence width.—Approximately 10.8 cm.
Individual flowers:
   Flower type.—Single, orchidaceous (zygomorphic), typical for Dendrobium.
   Rate of opening.—After the first flower has opened, it takes approximately four weeks before the top flower opens.
   Shape.—orchidaceous (zygomorphic), typical for Dendrobium, with three sepals, two lateral petals and a labellum (third petal).
   Height.—Approximately 5.7 cm.
   Diameter.—Approximately 6.4 cm.
   Depth.—Approximately 3.4 cm.
   Fragrance.—No fragrance.
   Persistent?.—Non-persistent.
   Quantity of petals.—Average: 3 (two lateral petals and a labellum (=third petal)) Only the two lateral petals are described here as such.
Petals:
   Arrangement.—Oppositely placed on each side of the column.
   Length.—Approximately 3.5 cm.
   Width.—Approximately 3.2 cm.
   Shape.—Broad obovate, slightly curved.
   Apex.—Rounded.
   Base.—Cuneate.
   Margin.—Entire.
   Appearance.—Both sides matte, velvety.
   Texture, upper forward surface.—glabrous, velvety.
   Texture, lower back surface.—glabrous, velvety.
   Color.—When opening, forward surface: White; near RHS NN155D, base red-purple; near RHS 72B. When opening, back surface: White; near RHS NN155D, base slightly darker; near RHS NN155A, outer base red-purple; near RHS 72B. Fully Opened, forward surface: White; near RHS NN155D, base red-purple; near RHS 72B to 72C. Fully Opened, back surface: White; near RHS NN155D, base slightly darker; near RHS NN155A, outer base red-purple; near RHS 72B to 72C Fading, forward surface: not fading (colours similar to “Fully Opened”) Fading, back surface: not fading (colours similar to “Fully Opened”).
   Color venation fully opened, forward surface.—As surface (colours similar to “Fully Opened”).
   Color venation fully opened, back surface.—As surface (colours similar to “Fully Opened”).
Labellum:
   Shape.—Deltoid, three-lobed, strongly involute.
   Length.—Approximately 2.8 cm.
   Width.—Approximately 2.1 cm (when “folded open”, approximately 3.4 cm).
   Texture.—glabrous, velvety.
   Appearance.—Matte, moderately velvety.
   Tip.—Rounded.
   Base.—Broad cuneate.
   Color.—When opening, forward surface: White; near RHS NN155C, base slightly darker; near RHS 155C. When opening, back surface: White; near RHS NN155C, base slightly darker; near RHS 155C. Fully Opened, forward surface: White; near RHS NN155C, base slightly darker; near RHS 155C. Fully Opened, back surface: White; near RHS NN155C, base slightly darker; near RHS 155C. Fading, forward surface: (colours similar to “Fully Opened”) Fading, back surface: (colours similar to “Fully Opened”).
   Color venation fully opened, forward surface.—As surface (colours similar to “Fully Opened”).
   Color venation fully opened, back surface.—As surface (colours similar to “Fully Opened”).
   Margin.—Entire.
   Callosities.—Dimensions: Average: 1.8 cm long and 0.5 cm wide Location: central placed on the labellum, coloured yellow-green; near RHS 150D and lighter.
Sepals:
   Quantity.—Average: 3.
   Appearance.—Matte, velvety.
   Length.—Approximately 3.5 cm.
   Width.—Dorsal sepal, approximately 1.6 cm. Lateral sepals, approximately 1.9 cm.
   Shape.—Dorsal sepal elliptic to oblong. Lateral sepals ovate, falcate.
   Apex.—Acute.
   Base.—Broad cuneate.
   Margin.—Entire.
   Texture.—Glabrous, velvety.
   Color.—Forward surface: White; near RHS NN155D Back surface: White; near RHS NN155D.
Pedicels:
   Length.—Approximately 3.2 cm.
   Diameter.—Approximately 0.2 cm.
   Angle.—Approximately 60° angle to peduncle (=0°).
   Strength.—Moderately strong.
   Texture.—Glabrous, moderately glossy.
   Color.—Green-white; near RHS 157A, base yellow-green; near RHS 144C.
Reproductive organs:
Stamens, style and stigma fused into column?: Yes
Column length: Average: 0.6 cm
Column diameter: Average: 0.8 mm
Column color: Green-white; near RHS 157D
Ovary:
   Position.—Superior, placed directly above stigma.
   Diameter.—Average: 0.2 cm.
   Color.—Green-white; near RHS 157A.
Stigma:
   Shape.—Clavate.
   Color.—Green-white; near RHS 157A.
   Dimensions.—Average: 0.3 cm high and 0.3 cm wide.
Pollinia:
   Color.—Yellow-orange; near RHS 14A.
   Dimensions.—Average: 0.075 cm long and 0.1 cm wide.
   Ouantity.—2.
Other characteristics:
Disease and pest resistance: High tolerance for typical diseases and pests of Dendrobium
Drought tolerance and cold tolerance: Normal for Genus.
Fruit/seed production: No fruits/seeds detected to date

What is claimed is:

1. A new and distinct cultivar of Dendrobium plant named ‘SPCDW1004’ as herein illustrated and described.
